(19) United States
(12) Patent Application Publication
(10) Pub. No.: US 2021/0104315 A1
(43) Pub. Date: Apr. 8, 2021

(54) ENGAGEMENT FOR INDIVIDUALS WITH DISABILITIES

(71) Applicant: MEMORE, Alexandria, VA (US)

(72) Inventor: Kathryn Callahan, Alexandria, VA (US)

(21) Appl. No.: 17/066,112

(22) Filed: Oct. 8, 2020

Related U.S. Application Data
(60) Provisional application No. 62/912,276, filed on Oct. 8, 2019.

(51) Int. Cl. G16H 40/20; G06Q 10/10; G06Q 30/00; G06Q 50/00; G06Q 10/06

(52) U.S. Cl. CPC G16H 40/20; G06Q 10/1093; G06Q 10/063118; G06Q 50/01; G06Q 30/0185

(57) ABSTRACT

The present application is directed to techniques that provide meaningful engagement for an individual with disabilities (Imp) living in residential care settings. The techniques increase personal satisfaction for an IWD and their families, improve culture within the residential care community, decrease health care costs, and lead to more effective use of care resources. The techniques provide interactive schedules to care staff and loved ones of an IWD that facilitate interactions, such as video conferences, without interfering with the schedule of the residential care community.

### CROSS REFERENCE TO RELATED APPLICATIONS

This application claims priority to and is based on U.S. Patent Application No. 62/912,276, filed Oct. 8, 2019, entitled “Engagement for Individuals with Disabilities,” the entire disclosure of which is incorporated herein by reference.

### TECHNICAL FIELD

The present disclosure is directed to a communication solution and, in particular, to a communication solution that provides assistive technology (AT) for an individual with disabilities (referred to herein as “IWD”) for whom activities of daily living (ADL)s, use of communication technology, and social isolation are known challenges.

### BACKGROUND

An IWD that is living in residential care settings often has limited options for interacting or communicating with friends and family aside from in-person visits. Thus, systems or techniques that facilitate interactions and communications (e.g., video chat sessions or messaging) between an IWD and his or her loved ones are desired, A non-exhaustive list of the disabilities that an IWD may live with are: Alzheimer's Disease or related dementias, Autism spectrum disorders, Cerebral Palsy, developmental disabilities, emotional or behavioral disabilities, intellectual disabilities, spinal cord injury and/or traumatic brain injury.

### SUMMARY

The present application is directed to techniques that provide meaningful engagement for an IWD. The techniques increase personal satisfaction for an IWD and his or her family, improve community culture, decrease health care costs, and lead to more effective use of a residential care community's resources. The techniques may be embodied in a system, apparatus, computer readable media, and the like.

For example, according to at least one embodiment, the present application is directed to a method for facilitating interactions for individuals with disabilities living in a residential care setting. The method includes generating scheduling slots or scheduling blocks during which a family user may schedule a call with an IWD and publishing the scheduling slots or scheduling blocks to a user device of the family user. Upon receiving a scheduling request from the user device, an alert is generated on one or more community devices associated with the residential care setting, the alert providing an indication of at least a particular scheduling slot or a particular scheduling block requested by the scheduling request. After the scheduling request is accepted, an interaction is deployed for the IWD during the particular scheduling slot or the particular scheduling block.

In some embodiments of this method, the publishing provides the user device of the family user with access to the scheduling slots or scheduling blocks without allowing the user device to access computing resources of the residential care setting. Additionally or alternatively, the one or more community devices can accept the scheduling request without interacting directly with the user device of the family user. Still further, in some embodiments of this method, the generating is based on: (1) user input that is input via the one or more community devices; (2) calendar data obtained from the one or more community devices; or (3) a combination of (1) and (2). In some instances, the method also determines if the family user associated with the user device is an authorized user and automatically accepts a scheduling request from the user device. Alternatively, the method may receive an acceptance of the scheduling request from a device of the one or more community devices.

In some embodiments of this method, an alert is sent to the user device once the scheduling request has been accepted. Additionally or alternatively, the interaction may only be deployable with a care staff member from the residential care setting present. In some of these instances, a particular care staff member from the residential care setting is assigned to the interaction prior to deploying the interaction, and an assignment is based on at least schedule data indicative of work schedules of care staff members at the residential care setting. Moreover, some embodiments may record interaction data of any completed interactions, and the assigning may also be based on recorded interaction data from previous interactions of the IWD. Alternatively, one or more notifications may be sent to the one or more community devices in response to receiving the scheduling request, the one or more notifications requesting a particular care staff member from the residential care setting to deploy the interaction.

Still further, in some embodiments, deploying the interaction includes initiating a call on a particular community device of the one or more community devices or sending an alert to a community device of the one or more community devices associated with a particular care staff member from the residential care setting who is assigned to the interaction.

### BRIEF DESCRIPTION OF THE DRAWINGS

FIG. 1 is a flow chart illustrating a method for facilitating interactions with an IWD living in a residential care setting, according to an example embodiment of the communication techniques presented herein.

FIG. 2 is a sequence diagram illustrating operations performed during the method of FIG. 1, according to an example embodiment of the techniques presented herein.

FIG. 3 is a sequence diagram demonstrating scheduling operations performed by the communication solution presented herein, according to an example embodiment.

FIG. 4 is a diagram illustrating how the communication solution presented herein may assign a scheduled interaction to specific resources, according to an example embodiment.

FIG. 5 is a diagram that demonstrates how an interaction might be initiated with the communication solution presented herein, according to an example embodiment.

FIG. 6 is a block diagram depicting a computer system upon which the techniques presented herein may be implemented, according to an example embodiment.

Like numerals identify like components throughout the figures.

### DETAILED DESCRIPTION

Overall, the techniques presented herein provide a communication solution that allows an IWD to regularly and easily communicate with anyone. Often, an IWD in a residential care setting does not have access to technology that allows for audio calls, video conferences, etc. with people outside of their care community (referred to herein as a “Community”). Thus, an IWD's interactions with friends and family may be limited to intermittent in-person visits. This increases the risk for boredom and social isolation for the IWD, thereby decreasing quality of life. In some instances, it may be possible for a loved one to befriend a staff member in the Community and communicate with an IWD through this staff member, but this may place an undue burden on the staff member and/or be unreliable due to the staff member's schedule, vacation time, regular workload, etc. The communication solution presented herein resolves this issue by allowing friends and family to schedule interactions, such as audio calls or video conferences, with an IWD residing in a Community in an automated manner. This eases the burden on care staff members without disrupting the daily schedule within a Community.

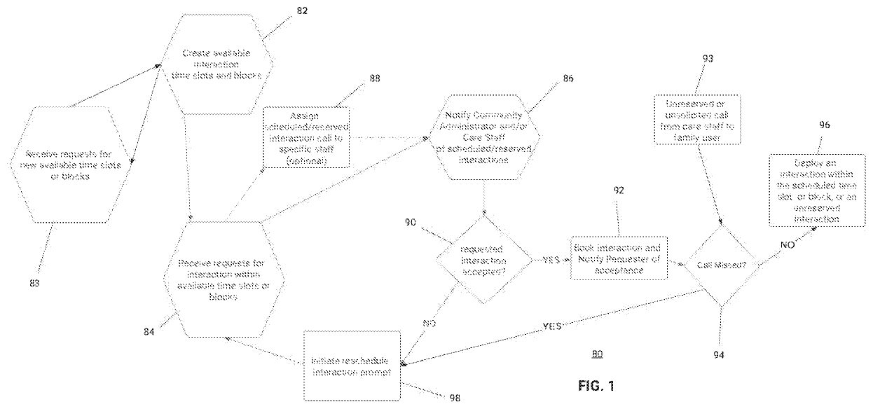

FIG. 1 illustrates a method 80 for scheduling an audio call or video conference with an IWD residing in a Community (e.g., an individual residing in a residential community setting). Generally, the communication solution may be implemented on/executed by a computing apparatus, such as a server. The computing apparatus may be local to a Community or remote from a Community and may have connectivity to wireless networks so that the computing apparatus can communicate with a variety of computing devices, such as smart phones, tablets, personal computers, smart televisions, etc. An example computing device on which the techniques presented herein may be implemented is described below in connection with FIG. 7.

Initially, at 82, the communication solution sets available times for interactions. In at least some embodiments, a Community Administrator (e.g., the person or persons that is authorized to assign user permissions at a one or more specific Communities, enable residents to use the communication solution, and schedule interactions within available times) creates time slots within blocks of time during which IWD residents may be available for interactions. For example, the Community Administrator may create time slots via a graphical user interface displayed on the computing device executing the communication solution or a graphical user interface displayed on a computing device (e.g., a tablet) communicating with the communication solution. Alternatively, the Community Administrator could upload a calendar to the communication solution and the communication solution may generate scheduling slots based on calendar data (e.g., slots in unfilled time periods). Regardless, the time slots and blocks of time available for interactions may, if desired, repeat on a daily basis, a weekly basis, monthly basis, etc. Additionally, in at least some instances, time slots can be created specifically for a particular Community or can be created to span multiple Communities (e.g., if multiple Communities operated by the same organization).

In at least some instances, the communication solution may allow requests for new time slots at 83. For example, requests can be submitted by friends and family of the IWD (referred to herein as “Family Users”) that are authorized to reserve time slots, or time blocks. Additionally or alternatively, the communication solution may, at 84, receive requests for interactions within one of the interaction time slots set at 82. When a Family User reserves a time slot or time block at 84, the communication solution alerts the Community Administrator of the reservation request at 86. That is, at 86, the communication solution may generate an alert on a community device connected to the communication solution. If the Community Administrator accepts the reservation request at 90, the communication solution fills that time slot and alerts the Family User of the acceptance at 92.

Alternatively, in some instances, when a Family User requests a reservation time slot or time block, the reservation request may trigger the communication solution to assign a care staff member to the time slot within the blocks automatically at 88. In some of these instances, a care staff member may be selected based on work schedules uploaded into the system (e.g., as calendar data), which may indicate when certain care staff members are busy, available, etc. Alternatively, at 88, the communication solution may alert a Community Administrator that an interaction has been scheduled and prompt the Community Administrator to assign a care staff member to the interaction. Still further, in sonic instances, the communication solution may push a reservation request to all care staff members at a Community at 88 and allow any care staff member to accept the reservation request.

Regardless of how or whether a care staff member is assigned to the interaction at 88, once an interaction is booked at 92, the communication solution will deploy the interaction at the scheduled time, as indicated at 96, unless the interaction is declined or canceled at 94, as is discussed in further detail below. In some instances, the deployment initiated at 96 may involve initiating an audio call or video conference (including audio and video) on a device associated with the specific care staff member assigned to the interaction. Alternatively, deploying the interaction at 96 may involve sending alerts or reminders to the specific care staff member assigned to the interaction, for example, so that the specific care staff member knows he/she should retrieve Community technology required for the interaction and bring it to the IWD resident. Still further, in some instances, an interaction might be commenced at 96 by powering on, starting, and/or unlocking a computing device (e.g., an IWD or community device) that is already positioned in the IWD resident's room. For example, in some instances, tablets (or other such computing devices) could be permanently assigned to an IWD resident, or an IWD resident's room, but only activated or unlocked for a booked interaction. In another instance, if a care staff member receives a reservation a day in advance, the care staff member may place a tablet (or other such device) in an IWD resident's room that morning. In some instances, the tablet (or other such device) may only unlock during a booked interaction (e.g., at 96). Additionally or alternatively, the tablet may lock out all functionality except for allowing an IWD resident to answer a call (insofar as “call,” when used alone herein refers to audio and/or video calls) from the Family User scheduled in a booked time slot, and answering deploys the interaction.

In some instances, Family Users may elect to include additional Family Users in an interaction (e.g. video call or messaging), thus creating a multi-person or group connection. Whether the interaction is one-to-one or one-to-many, the communication deployed at 96 will, among other advantages, enable families to be virtually present for meetings with care staff members, administrators in the Community, etc. and/or for appointments with nurses, physicians, or other medical professionals at the Community. Thus, the communication solution presented herein will allow Family Users to be present for appointments that they might not otherwise be able to attend—and may keep the Family Users up to date on the IWD resident's ongoing plan of care. For such meetings and appointments, a Community Administrator may invite pre-approved Family Users to the event. In fact, in some instances, the time slots generated (at 82) for specific Family Users may be customized based on a schedule of meetings and appointments of a particular IWD associated with the Family Users.

In some instances, interaction requests may be declined (by a Community or a Family User). Additionally or alternatively, interaction reservations might be cancelled, rescheduled or declined (e.g., not fulfilled at the scheduled time). If the Community Administrator declines a reservation request at 90, the communication solution may initiate rescheduling prompt at 98. The rescheduling prompt may request the Community Administrator to suggest a new time slot to the Family User or may automatically suggest a new time at 98 (e.g., to a scheduled appointment for the IWD associated with the Family User). Likewise, if the communication solution determines, at 94, that a booked interaction has been or should be declined, cancelled, or otherwise missed by. Family User or by Care Staff (e.g., as the result of either the assigned staff member, or the IWD resident's unavailability) the communication solution can initiate rescheduling prompt at 98. For example, if an IWD appointment is moved, the communication solution may decline a call and initiate rescheduling at 94 and 98, respectively. However, this implementation is not intended to be limiting and in other embodiments, calls missed or declined by a Family User may not be automatically rescheduled.

Still further, in some embodiments, the communication solution may allow non-reserved/unreserved or unsolicited calls to be made to a Family User, as is depicted at 93. If said Family User declines the call at 94, communication solution may alert the Family User of suggested rescheduling at 98

Notably, during method 80, a Family User does not obtain access to a Community's schedule or the Community Administrator, and care staff members need not interact directly with a Family User. Thus, the Community's schedule can remain secure and protected and the care staff member can retain some level of isolation from Family Users in the event that it would avoid interpersonal conflicts. In fact, in some instances, scheduling requests might be anonymized so as to prevent staff members from developing negative feelings towards Family Users who are constantly scheduling or rescheduling interactions. Moreover, the method 80 ensures that an IWD resident does not obtain unmonitored access to a phone or tablet that might result in unintended uses or that might expose the IWD resident to potential problems (e.g., scammers, telemarketers, etc.).

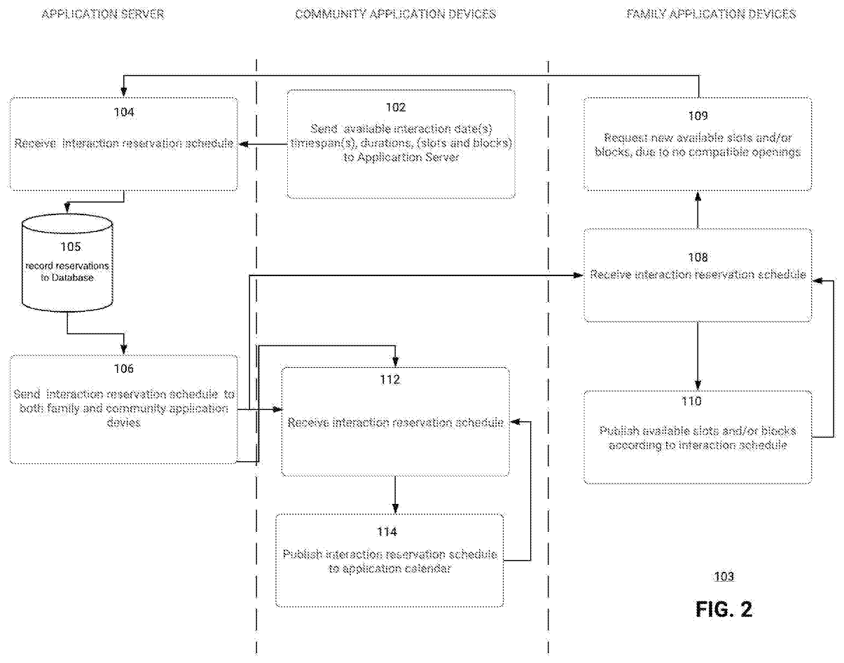

FIG. 2 is a sequence diagram 103 illustrating operations performed during the method 80 of FIG. 1. Initially, at 102, a Community Administrator creates time slots and blocks of time during which IWD residents may be available for interactions. As mentioned, the Community Administrator can create these time slots via a graphical user interface, a scheduling program or application, etc. For example, in some embodiments, the Community Administrators may use a dedicated Community Client Device at 102 to send available time slots to the community solution's Application Server. Moreover, in some embodiments, the Community Administrator, may input time slots and blocks of time available for interactions that repeat on a daily basis, a weekly basis, monthly basis, etc. unique to a specific Community or that span across multiple Communities within an Organization (e.g. the governor of a group of communities)

In order to maximize limited Community time and resources, the communication solution must set available times for care staff members to facilitate interactions such as date(s) timespan(s), duration, and quantity of available interactions within a timespan—this allows the interactions (e.g., video chat sessions) to integrate with staff workflow and existing staff and resident routines. As such, at 104 the Application Server receives or determines the date(s), timespan(s), duration, and quantity of available interactions from the time slots uploaded at 102 and records the information to a Schedule in the Database at 105. At 106, the application server retrieves the Schedule from the Database and sends the Schedule to the Community and family User client devices at 112 and 108 respectively. If the shared time slots are not compatible with the Family User's availability, then they can re-request new time slots in 109. In some embodiments, the application server sends the schedule to multiple Communities across an Organization at 106. That is, 108 and 112 may be representative of multiple Communities and Family Users associated with multiple communities.

Upon receiving the interaction reservation schedule at 108, a family application device publishes available date(s) and timespan of available calls to permissioned/authorized Family User client devices. Similarly, upon receiving the interaction reservation schedule at 112, a community application device publish available date(s) and timespan of available calls to care staff members so that care staff members are aware of interaction time slots and can provide updates if their personal schedule or the Community schedule changes. However, each of these steps may be iterative to ensure that the schedules published at 110 and 114 are up-to-date or “live.” This may prevent double bookings, which will ensure that care staff members are not stretched too thin or unable to assist with interactions. Moreover, the communication between the application server and the Family User client devices as well as the communication between the application server and the community application devices may be two-way so that the application server can pass updates from Family Users client devices (e.g., booking requests) to Community application devices and can pass updates from Community application devices (e.g., acceptance of requests, schedule changes, etc.) to Family User client devices.

In some embodiments a Community may wish to allow Family Users to view all specific time slots (e.g., 9:00 AM, 10:00 AM, 11:00 AM, etc.), where in others, a Community may wish to allow Family Users to see only blocks of slots available for interactions (e.g., 8:00-11:00 AM). Thus, the application server may allow for toggling of such settings between different communities. Additionally, some Communities may publish booked time slots while other Communities may only continue to publish available time slots. For example, based on settings input or toggled by Community Administrators, one Community's schedule may show booked and available time slots published at 110 while another Community's schedule may show only time blocks that still have availability (without showing blocks of time that are booked to capacity). However, preferably, the schedule published at 114 may show all slots to the Community, perhaps even including potential time slots that were not input at 102 (e.g., schedule published at 114 may indicate that additional slots can be opened for booking if desired). In some instances, time slots may be suggested as additional slots if they are within certain hours (e.g., between 9:00 AM and 5:00 PM), but not selected at 102. Alternatively, time slots may be suggested as additional slots if the slots had been regularly used on that day, in past weeks, etc. This may help create a consistent schedule that is beneficial to IWDs.

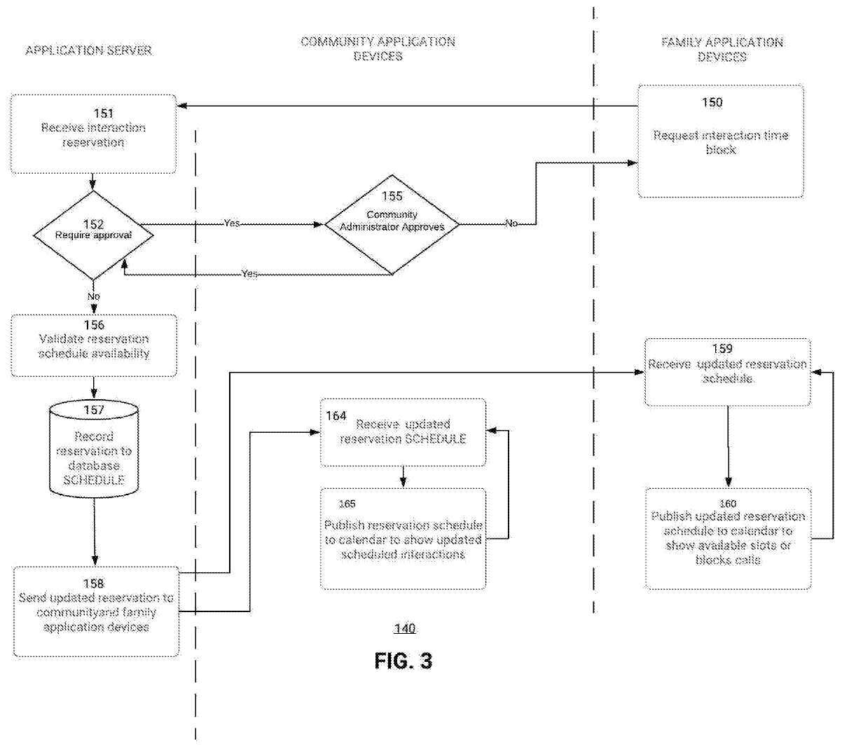

FIG. 3 is a sequence diagram 140 that demonstrates how authorized Family Users on a Family Application Device may reserve an interaction slot within a block of time, according to an example embodiment. Initially, at 150, a Family User selects a time slot on any date where blocks or slots have been made available by a Community Administrator. In the depicted embodiment, the Family User cannot see the slots of time within the block, and is therefore selecting a block of time at 150 (e.g., Wednesday AM). Then, the communication solution either assigns the reservation to a non-disclosed slot within the available block or allows a Community Administrator or care staff member to select a time slot within the block. Alternatively, a block may afford a Community employee flexibility in executing scheduled calls within the block of time, which is often beneficial to Community workflow. For example, in some instances, a care staff member can see the number of scheduled interactions but do not necessarily need to execute them in the order received/booked. That said, in other embodiments, a Family User may be able to reserve a specific slot on the schedule at 150. In fact, the option to publish availability in slot or block format may be critical to workflow in a care Community because it allows for accommodation of many varied Community and organizational operational schemas.

When a Family User reserves a time slot or time block at 150, the family user client device pushes the request to the application server executing the communication technology presented herein. After receiving a request at 151, the application server determines, at 152, if the request requires approval from Community. For example, the application server may analyze a lookup table to determine whether the Community to which the request pertains requires approvals for requests. If the Community at issue requires approvals, the application server alerts the Community Administrator of the reservation request at 155. The Community Administrator then approves or declines the reservation at 155. If the Community Administrator declines the reservation at 155, the Family User is notified on the Family Application Device. Although this arrow flowing from 155 to 150 appears to indicate that the Community Application device communicates directly with the family application device when a request is not approved, this communication may, in at least some embodiments, actually flow through the application server, as the application server may in essence, serve as the hub for interaction bookings. For example, if a Community Administrator declines a request on a Community application device, the Community application device may communicate this to the application server, which may push a rescheduling notification to the family application device. In at least some embodiments, when a request is declined, the Community Administrator may suggest a new time slot and the communication solution may alert the Family User of the suggested new time slot. Either way, after a request is declined, the Family User may restart the reservation process at 150.

If the Community Administrator approves the reservation request at 155 or an approval is not required, the application server executing the communication solution validates the reservation at 156, records valid reservations to the database at 157, and sends booking confirmations at 158. The validating at 156 may include determining that a user associated with an approved appointment is an authorized user for the IWD for which an interaction is booked, determining that the time slot remains available (e.g., is not already booked), and/or any other authorization or validation determinations. Once a requested interaction is booked, recorded, and published (i.e., sent out), the Community Application Devices and Family Application Devices will receive updated reservation schedules at 164 and 159, respectively, and the updated scheduled will be published at 165 and 160, respectively. Steps 164, 165, 159, and 160 may be similar to steps 112, 114, 108, and 110 discussed above. Thus, any description of steps 112, 114, 108, and 110 included above should be understood to apply to steps 164, 165, 159, and 160. Additionally, these steps may be completed iteratively if calls are rescheduled.

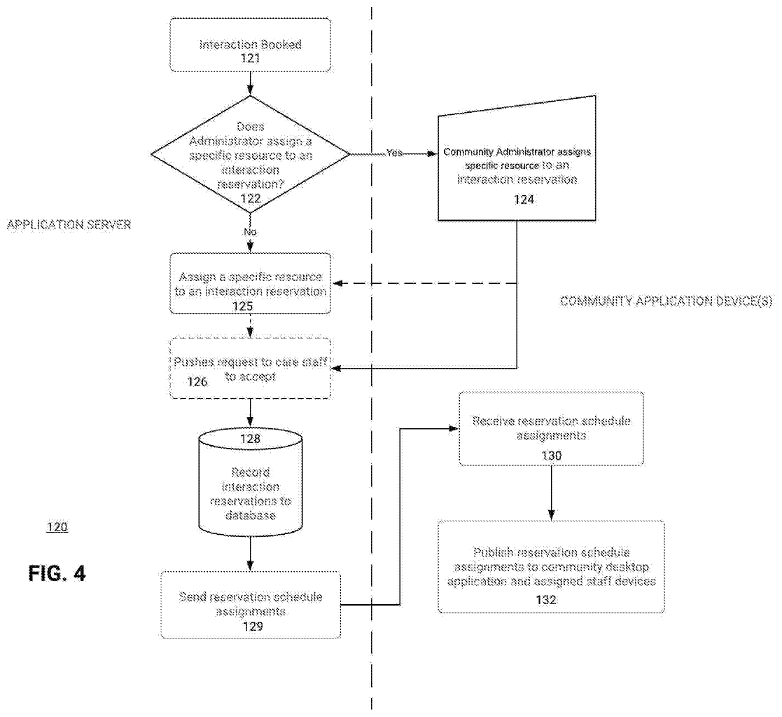

FIG. 4 is a diagram 120 that illustrates how the communication solution presented herein may assign a scheduled interaction to specific Community resources (insofar as resources includes staff and devices/equipment to facilitate interactions). That is, FIG. 4 illustrates operations of the communication solution after an interaction appointment has been requested and approved (e.g., in accordance with the methods and diagrams shown in FIGS. 1-3). Thus, diagram 120 begins at 121, when an interaction is booked. Once an interaction is booked at 120, the communication solution presented herein determines, at 122, whether an administrator will need to assign resources, such as a care staff member and video conference equipment, to an interaction. The communication solution may make this determination based on preferences of the specific Community in which the interaction will occur.

If a Community has indicated that an administrator should assign resources to an interaction reservation, the communication solution pushes a notification to the community application device at 122 and at 124, the Community Administrator assigns specific resources to the interaction reservation. For example, the Community Administrator many assign a care staff member and a tablet. If, at 122, the communication solution determines that a Community does not require its Administrator to assign resources, the Communication solution automatically assigns resources at 125. In some embodiments, automatic resource assignment may be a default setting, but in other embodiments, Administrator resource assignment may be a default setting. Moreover, in some embodiments, if the communication solution does not receive care staff assignments within a predefined time period (e.g., 24 hours), resources may be automatically assigned at 125 (as shown by a dashed arrow).

If the communication solution automatically assigns resources to an interaction reservation, the communication solution may consider a number of factors and/or rules in making the assignment. The factors may include scheduling data (including care staff members' work schedules (e.g., uploaded into the communication solution) and Community schedules), user preferences, past experiences (i.e., historical data), type of interaction (e.g., video call or audio call), and purpose of appointment (e.g., medical, routine hello, or care planning). For example, if a particular MD has conducted past interaction reservations with a particular care member, the communication solution may assign the care staff member to the reservation, provided the care staff member is available during the scheduled interaction. Additionally or alternatively, the communication solution may consider the Family User involved in the interaction and assign a care staff member based, at least in part, on the Family User. In fact, in at least some embodiments, Family Users, the IWD, and/or care staff members may be able to provide the communication solution with preferences lists. The Family User preference lists may indicate a preference for care staff members and the care staff member preference lists may indicate a preference for Family Users. Still further, in some embodiments, care staff members may be assigned to reserved interactions at or close to randomly, perhaps cycling through care staff members so that each care staff member facilitates an equal number of interactions.

Allowing for Family User preferences may be important for IWD interactions, since the IWD may often be less particular about a care staff member than a family user (e.g., since the family User may be more aware of previous interpersonal conflicts and other such experiences). On the other hand, in some Communities, IWD residents are bonded or paired with a particular care staff member and, thus, the communication solution must also consider IWD or care staff preferences. The assignments made at 125 may be especially useful where specific care staff members have developed bonds with IWD residents and/or Family Users, and/or if specific care staff members have unique abilities to facilitate meaningful interactions between certain IWD residents and Family Users since the communication solution may give preference to positive past experiences and/or routines. However, that all said, in some instances, a Community may choose to omit care staff member assignments and have predetermined care staff members assigned to all interactions for certain IWD residents or resident groups (i.e., certain residents can only have an interaction with a particular care staff member).

Still further, the communication solution may assign technology to an interaction reservation at 125 based on, among other factors, the type of interaction (e.g., video conference, call, medical appointment, etc.), and the Community schedule. For example, certain tablets may be associated with in-bedroom video conferences while other computing devices may be reserved for interactions scheduled to occur in rooms reserved for medical appointments. However, in some instances, Community may choose to omit assignments of interaction equipment (or other resources) and have interaction equipment assigned to specific IWD residents or resident groups for interactions.

Once interaction equipment, a care staff member, and any other resources are assigned to an interaction reservation, the communication solution may optionally push notifications to care staff members at 126. In some embodiments, this push notification may allow a care staff member to accept the assignment, but in other embodiments, the notification may simply notify the care staff member of a change in their schedule, without providing option for acceptance. Alternatively, the notification may be pushed to a plurality of care staff members at 126 and allow any care staff member to accept the request.

At 128, the application server records the assignment(s) to the Schedule. Then, at 129, the application server sends assignment(s) from the database to community application devices. The community application devices (e.g., smartphones, desktops, tablets, etc. associated with care staff members) receive the assignment(s) at 130 and publish the assignments at 132 so that care staff members can view their assignments or the assigned resources for a particular interaction.

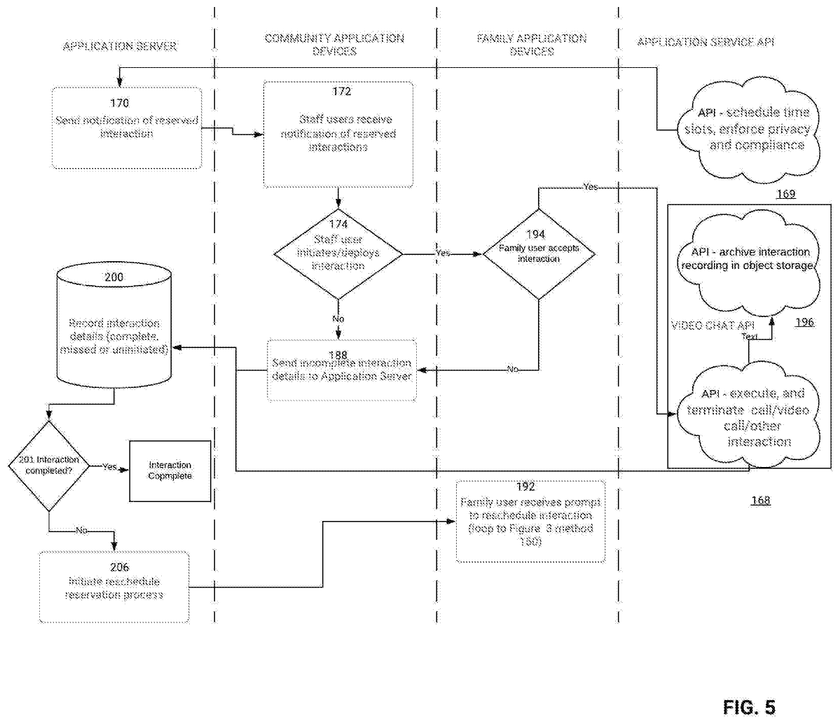

Now turning to FIG. 5, this Figure illustrates a diagram 168 that demonstrates how an interaction might be initiated in accordance with at least one example embodiment. In at least some embodiments, an interaction is prompted by sending a reminder to a care staff member. More specifically, in the depicted embodiment, the application service application programming interface (API) 169 sends the appropriate time slot reservation data to the application server. The application service sends a notification of a reserved interaction is sent from the application server to one or more community application devices at 170 via any combination of push notifications, entails, or other alerts, which may each be curated to target specific Community Application Devices based on assignments or IWD resident groups to allow receipt at 172. Resident groups may be created in any structure that supports the workflow of the Community (e.g. by room location, team lead, level of care, available time of day, etc.). Ultimately, it is critical that the notifications received at 172 fit seamlessly into the Community workflow so as to achieve the best outcomes for care staff members and an IWD, while ensuring that the IWD can easily interact with their loved ones.

Upon receiving the notification at 172, the care staff member will either retrieve the necessary equipment or bring the IWD to the equipment (e.g., if a tablet in the IWD's room will soon be unlocking/activating). Additionally, at this point, the care staff member can decide, based on the IWD resident's likelihood to participate in and benefit from the interaction, whether to accommodate the interaction reservation. For example, if an IWD is not in a good mental state and the care staff member believes an interaction might exacerbate their condition, the care staff member can use their judgment to determine whether the interaction should occur. Thus, it may be critical that a care staff member be involved in deployment of an interaction and that MD not be allowed to commence an interaction unsupervised.

In the depicted embodiment, the care staff member deploys the reserved interaction with the Family User at 174 and the Family User accepts the interaction (e.g., answers a call or video call) at 194. However, in other embodiments, a care staff member may prepare an IWD for an interaction, but the Family User may initiate the interaction. That said, it may be preferable for the IWD and care staff member to initiate an interaction so that the interaction fits seamlessly into a Community's schedule and workflow. Either way, once a call is initiated and accepted, an interaction API 196 may be utilized to execute and subsequently terminate the interaction. At the conclusion of the interaction, a recording of the session may be stored in object storage for archival purposes.

If, instead, a care staff member decides not to deploy an interaction or a deployed interaction is not accepted (e.g., if the care staff member deploys the reserved interaction with the Family User at 174 and the Family User does not answer), the device initiating the interaction may send details of the incomplete interaction to the Application Server. In the depicted embodiment, the community application device sends the details of the incomplete interaction at 188 and the interaction details are recorded to the database at 200.

Interaction details (i.e., interaction data) are also recorded to database 200 during or at the end of an interaction. The interaction details may include high-level details, such as the names of the parties involved (e.g., the names of the IWD, the care staff member, and the family user), the length of the interaction, the type of interaction (e.g., phone or video), the purpose of appointment (e.g., medical, routine hello, or care planning). Additionally, the interaction details might include more detailed information (referred to as reaction data), such as the IWD's emotional response to the interaction, level of recall during the interaction, etc. The high-level interaction details will likely be available from the booking process, but the more detailed interaction details (and/or the high-level interaction details) can be obtained from manual data entries or using data collection technologies (e.g., emotional detection technologies) now known or developed hereafter. Additionally or alternatively, integrated surveys may be presented at termination of an interaction to inform potential improvements to the reservation and interaction deployment process specific to the IWD resident and the Community.

After the interaction details are recorded at 200, the communication solution determines if an interaction needs to be rescheduled. If an interaction is determined to be completed at 201, for example, based on a length of the interaction and the method of termination (e.g., the interaction was not ended due to a lost connection), the communication solution may deem the interaction complete. lf, instead, the call was incomplete or ended prior to completion (e.g., due to a technical difficulty or a scheduling issue), the call can be rescheduled at 206. Upon initiating of rescheduling at 206, the application server can prompt the Family User to reschedule the interaction. At 192, the Family User application device will receive the notification. Thus, in the event that a Family User misses an interaction that was initiated by Care Staff or a Community Administrator whether previously scheduled or otherwise, 206 will not prompt the Family User to reschedule the interaction

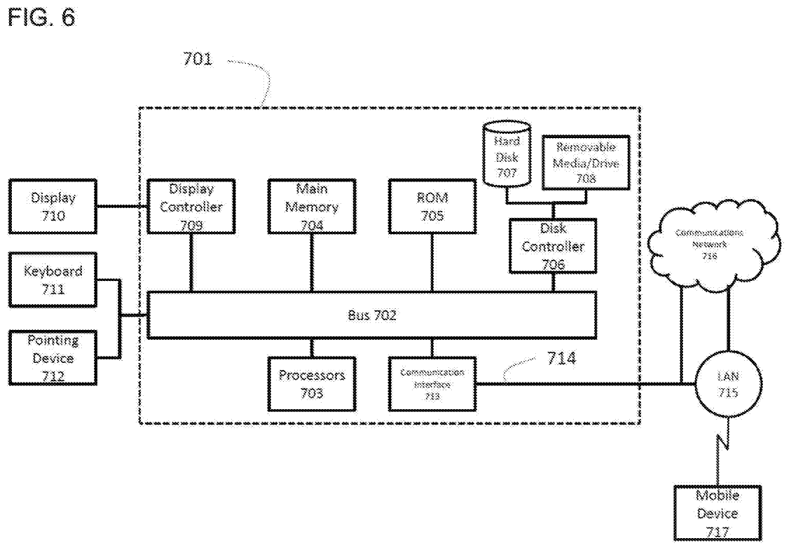

Now referring to FIG. 6 for a description of a computer system 701 upon which the techniques presented herein may be implemented. The computer system 701 may be representative of the application server illustrated and described herein as well as any other device that might execute the communication solution presented herein. Additionally, computer system 701 might be representative of the Community application devices, and/or family application devices illustrated and described herein.

The computer system 701 includes a bus 702 or other communication mechanism for communicating information, and a processor 703 coupled with the bus 702 for processing the information. While the figure shows a single block 703 for a processor, it should be understood that the processors 703 represent a plurality of processing cores, each of which can perform separate processing. The computer system 701 also includes a main memory 704, such as a random access memory (RAM) or other dynamic storage device (e.g., dynamic RAM (DRAM), static RAM (SRAM), and synchronous DRAM (SD RAM)), coupled to the bus 702 for storing information and instructions to be executed by processor 703. In addition, the main memory 704 may be used for storing temporary variables or other intermediate information during the execution of instructions by the processor 703.

The computer system 701 further includes a read only memory (ROM 705 or other static storage device (e.g., programmable ROM (PROM), erasable PROM (EPROM), and electrically erasable PROM (EEPROM)) coupled to the bus 702 for storing static information and instructions for the processor 703.

The computer system 701 also includes a disk controller 706 coupled to the bus 702 to control one or more storage devices for storing information and instructions, such as a magnetic hard disk 707, and a removable media drive 708 (e.g., floppy disk drive, read-only compact disc drive, read/write compact disc drive, tape drive, and removable magneto-optical drive, optical drive). The storage devices may be added to the computer system 701 using an appropriate device interface (e.g., small computer system interface (SCSI), integrated device electronics (IDE), enhanced-IDE (E-IDE), direct memory access (DMA), or ultra-DMA).

The computer system 701 may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)), that, in addition to microprocessors and digital signal processors may individually, or collectively, are types of processing circuitry. The processing circuitry may be located in one device or distributed across multiple devices.

The computer system 701 may also include a display controller 709 coupled to the bus 702 to control a display 710, such as liquid crystal display (LCD), or a light emitting diode (LED) display, for displaying information to a computer user. The computer system 701 includes input devices, such as a keyboard 711 and a pointing device 712, for interacting with a computer user and providing information to the processor 703. The pointing device 712, for example, may be a mouse, a trackball, or a pointing stick for communicating direction information and command selections to the processor 703 and for controlling cursor movement on the display 710. The pointing device 712 may also be incorporated into the display device as, for example, a capacitive touchscreen, and/or a resistive touchscreen.

The computer system 701 performs a portion or all of the processing steps of the invention in response to the processor 703 executing one or more sequences of one or more instructions contained in a memory, such as the main memory 704. Such instructions may be read into the main memory 704 from another computer readable medium, such as a hard disk 707 or a removable media drive 708. One or more processors in a multi-processing arrangement may also be employed to execute the sequences of instructions contained in main memory 704. In alternative embodiments, hard-wired circuitry may be used in place of or in combination with software instructions. Thus, embodiments are not limited to any specific combination of hardware circuitry and software.

As stated above, the computer system 701 includes at least one computer readable medium or memory for holding instructions programmed according to the embodiments presented, for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SD RAM, or any other magnetic medium, compact discs (e.g., CD-ROM), or any other optical medium, punch cards, paper tape, or other physical medium with patterns of holes, or any other medium from which a computer can read.

Stored on any one or on a combination of non-transitory computer readable storage media, embodiments presented herein include software for controlling the computer system 701, for driving a device or devices for implementing the invention, and for enabling the computer system 701 to interact with a human user (e.g., a network engineer). Such software may include, but is not limited to, device drivers, operating systems, development tools, and applications software. Such computer readable storage media further includes a computer program product for performing all or a portion (if processing is distributed) of the processing presented herein.

The computer code devices may be any interpretable or executable code mechanism, including but not limited to scripts, interpretable programs, dynamic link libraries (DLLs), Java classes, and complete executable programs. Moreover, parts of the processing may be distributed for better performance, reliability, and/or cost. Such distributed parts may leverage Platform-as-a-Service or other cloud computing technologies.

The computer system 701 also includes a communication interface 713 coupled to the bus 702. The communication interface 713 provides a two-way data communication coupling to a network link 714 that is connected to, for example, a local area network (LAN) 715, or to another communications network 716 such as the Internet. For example, the communication interface 713 may be a wired or wireless network interface card to attach to any packet switched (wired or wireless) LAN. As another example, the communication interface 713 may be an asymmetrical digital subscriber line (ADSL) card, an integrated services digital network (ISDN) card or a modem to provide a data communication connection to a corresponding type of communications line. Wireless links may also be implemented. In any such implementation, the communication interface 713 sends and receives electrical, electromagnetic, or optical signals that carry digital data streams representing various types of information.

The network link 714 typically provides data communication through one or more networks to other data devices. For example, the network link 714 may provide a connection to another computer through a local area network 715 (e.g., a LAN) or through equipment operated by a service provider, which provides communication services through a communications network 716. The local network 714 and the communications network 716 use, for example, electrical, electromagnetic, or optical signals that carry digital data streams, and the associated physical layer (e.g., CAT 5 cable, coaxial cable, optical fiber, etc.). The signals through the various networks and the signals on the network link 714 and through the communication interface 713, which carry the digital data to and from the computer system 701 maybe implemented in baseband signals, or carrier wave based signals. The baseband signals convey the digital data as unmodulated electrical pulses that are descriptive of a stream of digital data bits, where the term “bits” is to be construed broadly to mean symbol, where each symbol conveys at least one or more information bits. The digital data may also be used to modulate a carrier wave, such as with amplitude, phase, and/or frequency shift keyed signals that are propagated over a conductive media, or transmitted as electromagnetic waves through a propagation medium. Thus, the digital data may be sent as unmodulated baseband data through a “wired” communication channel and/or sent within a predetermined frequency band, different than baseband, by modulating a carrier wave. The computer system 701 can transmit and receive data, including program code, through the network(s) 715 and 716, the network link 714 and the communication interface 713. Moreover, the network link 714 may provide a connection through a LAN 715 to a mobile device 717 such as a personal digital assistant (PDA) laptop computer, or cellular telephone.

What is claimed is:

1. A method for facilitating interactions for individuals with disabilities (IWDs) living in a residential care setting, comprising:
generating scheduling slots or scheduling blocks during which a family user may schedule a call with an IWD;
publishing the scheduling slots or scheduling blocks to a user device of the family user;
upon receiving a scheduling request from the user device, generating an alert on one or more community devices associated with the residential care setting, the alert providing an indication of at least a particular scheduling slot or a particular scheduling block requested by the scheduling request; and
after the scheduling request is accepted, deploying an interaction for the IWD during the particular scheduling slot or the particular scheduling block.

2. The method of claim 1, wherein the publishing provides the user device of the family user with access to the scheduling slots or scheduling blocks without allowing the user device to access computing resources of the residential care setting.

3. The method of claim 1, wherein the one or more community devices can accept the scheduling request without interacting directly with the user device of the family user.

4. The method of claim 1, wherein the generating is based on: (1) user input that is input via the one or more community devices; (2) calendar data obtained from the one or more community devices; or (3) a combination of (1) and (2).

5. The method of claim 1, further comprising:
determining the family user associated with the user device is an authorized user; and
automatically accepting a scheduling request from the user device.

6. The method of claim 1, further comprising:
receiving an acceptance of the scheduling request from a device of the one or more community devices.

7. The method of claim 1, further comprising:
sending an alert to the user device once the scheduling request has been accepted.

8. The method of claim 1, wherein the interaction is only deployable with a care staff member from the residential care setting present.

9. The method of claim 8, further comprising:
assigning a particular care staff member from the residential care setting to the interaction prior to deploying the interaction, wherein an assignment is based on at least schedule data indicative of work schedules of care staff members at the residential care setting.

10. The method of claim 9, further comprising:
recording interaction data of any completed interactions,
wherein the assigning is also based on recorded interaction data from previous interactions of the IWD.

11. The method of claim 8, further comprising:
sending one or more notifications to the one or more community devices in response to receiving the scheduling request, the one or more notifications requesting a particular care staff member from the residential care setting to deploy the interaction.

12. The method of claim 1, wherein deploying the interaction comprises:
initiating a call on a particular community device of the one or more community devices; or
sending an alert to a community device of the one or more community devices associated with a particular care staff member from the residential care setting who is assigned to the interaction.

13. A computing apparatus for facilitating interactions for individuals with disabilities (IWDs) living in residential care settings, the communication solution, comprising:
one or more network interface units configured to enable network connectivity to a network;
a memory storing instructions; and
a processor configured to:
generate scheduling slots or scheduling blocks during which a family user may schedule a call with an IWD;
publish the scheduling slots or scheduling blocks to a user device of the family user;
upon receiving a scheduling request from the user device, generate an alert on one or more community devices associated with the residential care setting, the alert providing an indication of at least a particular scheduling slot or a particular scheduling block requested by the scheduling request; and
after the scheduling request is accepted, deploy an interaction for the IWD during the particular scheduling slot or the particular scheduling block.

14. The computing apparatus of claim 13, wherein publishing provides the user device of the family user with access to the scheduling slots or scheduling blocks without allowing the user device to access computing resources of the residential care setting.

15. The computing apparatus of claim 13, wherein the one or more community devices can accept the scheduling request without interacting directly with the user device of the family user.

16. The computing apparatus of claim 13, wherein generation of the scheduling slots or the scheduling blocks is based on: (1) user input that is input via the one or more community devices; (2) calendar data obtained from the one or more community devices; or (3) a combination of (1) and (2).

17. The computing apparatus of claim 13, wherein the processor is further configured to:
determine the family user associated with the user device is an authorized user and automatically accept a scheduling request from the user device; or
receive an acceptance of the scheduling request from a device of the one or more community devices.

18. The computing apparatus of claim 13, wherein the processor is further configured to:
assign a particular care staff member from the residential care setting to the interaction prior to deploying the interaction, wherein an assignment is based on at least schedule data indicative of work schedules of care staff members at the residential care setting.

19. The computing apparatus of claim 13, wherein, to deploy, the processor is configured to:
initiate a call on a particular community device of the one or more community devices; or
send an alert to a community device of the one or more community devices associated with a particular care staff member from the residential care setting who is assigned to the interaction.

20. One or more non-transitory computer readable storage media encoded with software comprising computer executable instructions and when the software is executed operable to:
generate scheduling slots or scheduling blocks during which a family user may schedule a call with an individuals with disabilities (IWD) living in a residential care setting;
publish the scheduling slots or scheduling blocks to a user device of the family user;
upon receiving a scheduling request from the user device, generate an alert on one or more community devices associated with the residential care setting, the alert providing an indication of at least a particular scheduling slot or a particular scheduling block requested by the scheduling request; and
after the scheduling request is accepted, deploy an interaction for the IWD during the particular scheduling slot or the particular scheduling block.
